(19) United States
(12) Patent Application Publication
(10) Pub. No.: US 2020/0347430 A1
(43) Pub. Date: Nov. 5, 2020

(54) METHODS AND COMPOSITIONS FOR IMPROVING DETECTION OF MICROORGANISMS

(71) Applicant: BactoByte Ltd., Jerusalem (IL)

(72) Inventor: Vladimir GLUKHMAN, Jerusalem (IL)

(73) Assignee: BactoByte Ltd., Jerusalem (IL)

(21) Appl. No.: 16/931,469

(22) Filed: Jul. 17, 2020

Related U.S. Application Data
(63) Continuation of application No. PCT/IL2019/050064, filed on Jan. 16, 2019.
(60) Provisional application No. 62/618,094, filed on Jan. 17, 2018.

(51) Int. Cl. C12Q 1/04; G01N 21/64; C12Q 1/24

(52) U.S. Cl. CPC C12Q 1/04; G01N 2021/6439; C12Q 1/24; G01N 21/6428

(57) ABSTRACT

The present invention discloses method and means for rapid detection and quantification of specific live and recovered microorganisms in a sample, comprising steps of contacting the sample with a detection cocktail, the detection cocktail comprises: (i) a nutrient medium comprising at least one of cell growth stimulators from potential-host of the pathogen for accelerated selective growing and multiplying of said microorganism; (ii) at least one fluorescent marker molecule for detection of intracellular metabolism by a sensor, (iii) at least one metabolism activator for specifically increasing intracellular metabolism of said specific microorganism and increasing intracellular concentration of said fluorescent marker molecule into said specific microorganism; (iv) determination of numerical range for the level of intracellular fluorescence intensity of the said marker for the differentiation of specific recovered microorganisms from all other objects and (v) determining recovered specific microbial cells by measurement of intra-cellular concentration of metabolism markers in each microscope visualized cell. The rapid detection and quantification of specific live and recovered microorganisms in a sample is further performed by: (i) measuring of fluorescent intensity of the marker molecules in individual cells by the sensor, while intensity levels correlated to predetermined metabolism level of said specific microorganism; (ii) determining the gray level to a predetermined threshold of metabolism; and (iii) further correlating the gray levels above the predetermined threshold with quantity of recovered high metabolic-active said microorganism in the sample.

### RELATED APPLICATIONS

This application is a Continuation of PCT Patent Application No. PCT/IL2019/050064 having International filing date of Jan. 16, 2019, which claims the benefit of priority under 35 USC § 119(e) of U.S. Provisional Patent Application No. 62/618,094 filed on Jan. 17, 2018. The contents of the above applications are all incorporated by reference as if fully set forth herein in their entirety.

### FIELD OF INVENTION

The current invention generally pertains to methods and compositions for improving detection of microorganisms.

More specifically, the present invention relates to a method for rapid detection of a number of live and recovered microorganisms in a sample by measuring intra-cellular metabolism of said microorganism, following contacting said sample with a specific detection cocktail which comprises a nutrient medium comprising host-derived fraction, for selective growing and accelerated multiplying of said microorganism; at least one fluorescent marker molecule of intra-cellular metabolism; and at least one metabolic activator for specifically increasing metabolism of said specific microorganism and increasing intra-cellular concentration of said fluorescent marker molecule into said specific microorganism; contacting said sample with said detection cocktail and determining recovered microbial cells by measurement of intracellular concentration of metabolism markers

### BACKGROUND OF INVENTION

Bacterial contamination and infection is a significant problem to public health, food, industry, environmental biosafety, and many other areas.

Microorganisms in Blood and in Sterile Fluids:

Human biological fluids are frequently obtained from patients showing symptoms of infection in the corresponding body area to isolate and identify the etiological agent. Samples of this kind are also taken to detect the presence of microorganisms in at-risk patients showing particular pathological conditions (patients subjected to abdominal surgery, ascites in cirrhotic patients, hematological disorders, etc.).

The recovery of microorganisms from blood is crucial for proper diagnosis and treatment of infection. To obtain accurate results, it is necessary to maximize the number of organisms collected from a given sample. This is challenging due to the fact that the concentrations of pathological organisms in the blood vary enormously. One example of this wide range of concentrations is the case of bacteremia, a condition where viable bacteria are present in the circulating blood.

The prompt diagnosis and treatment of bacteremia is of significant interest to health care professionals. When left undiagnosed, bacteremia can lead to systemic inflammatory response syndrome (SIRS) and patients are often at high risk for developing sepsis, the leading cause of death in critically ill patients in Europe and the U.S.A.

Sepsis is a life-threatening condition caused by the uncontrolled, systemic, inflammatory response to bacterial, viral or fungal infection. Sepsis represents a substantial health burden. The incidence of sepsis and the number of sepsis-related deaths are increasing, due to a variety of reasons attributed to the aging of the population, the increasing longevity of patients with chronic diseases, the increasingly aggressive cancer therapies and the increasing use of invasive devices, like cardiac pacemakers, valves and defibrillators, and procedures for a variety of medical conditions are as well as the widespread use of broad-spectrum antibiotics which has increased the rates of both antibiotic resistance and nosocomial fungal infections.

Consequently, it is necessary to develop quick and accurate diagnostics for detecting bacteria in blood, urine, and other normally sterile fluids. Furthermore, rapid and accurate identification of sepsis and its causative organisms are a prerequisite for successful therapy. Delayed recognition of sepsis and inappropriate initial antibiotic therapy are associated with an increase in mortality and morbidity. The current gold standard for the diagnosis of sepsis is culture of blood and other body fluids or tissues. However, even in severe sepsis, blood cultures yield the causative microorganism in only 20-40% of patients. Moreover, at least 24 hours are needed to get preliminary information about the potential organism.

Detection of Microorganisms in Pharmaceutical Preparations:

Traditional methods to assure quality and detect any microbial contaminants in intravenous products takes at least 2 weeks to complete. Hospital-prepared intravenous products are often high risk and have short shelf lives. This sometimes means that the traditional quality microbiological results are only available after the product has been administered to the patient. As a consequence, there have been some fatal incidents in which contaminated intravenous products have been used.

Detection of Foodborne Pathogens:

The detection and enumeration of pathogens in food and on surfaces that come into contact with food are an important component of any integrated program to ensure the safety of foods throughout the food supply chain. Both government authorities and food companies use microbiological analysis to monitor the state of contamination at all times and analyze its trends so as to detect emerging risks. Traditional culture methods for detecting microorganisms in food can be laborious and may require several days before results are known. Products that are minimally processed have an inherently short shelf life, which prevents the use of many of these conventional methods.

Waterborne Pathogens:

Waterborne disease is a global burden, while morbidity and mortality caused by contaminated water are enormous and need to be controlled by improving the security of drinking water.

Waterborne infections are caused by ingestion, airborne or contact with contaminated water by a variety of more than 1400 species of infectious agents which includes bacteria, viruses, protozoa fungi and helminths, which may lead to diarrhea, gastrointestinal diseases and systematic illnesses, and even death. It is estimated that 3.2% of deaths globally are attributable to unsafe water caused by poor sanitation and hygiene.

Detection methods play a major role in monitoring water quality, surveillance, and quantitative microbial risk assessment; thus, have a major influence on implementing the best practices to alleviate and prevent threats that allow achieving the goal of water safety.

Bacterial contamination and infection is a significant problem to public health, food, industry, environmental biosafety, and many other areas. However, current methods for detecting bacteria in medical, veterinary, agricultural, food processing, industrial and other contexts are slow, require specialized personnel or equipment to execute, and are often expensive. There is a large unmet need for technologies that can provide quick, sensitive, and specific detection of pathogens to enable proactive, convenient, and rapid safety programs that reduce costs and threats to human health.

### BRIEF DESCRIPTION OF THE FIGURES

FIG. 1: Intracellular concentration of marker molecule of cellular metabolism as a result of the influence of metabolism activators (Scheme)

Illustrations showing the effect of bacterial recovery or metabolism stimulators/activators on dyes concentrations of marker molecules in various cells: Marker molecules dyes increase their concentration in recoverable cells with increased metabolism. The concentration is low in non-recoverable cells: In non-growth and anabiotic microorganisms which have low level of metabolism, the dyes remain confined to the extracellular space.

FIG. 2: Morphology of tested Coliform bacteria

FIGS. 3A-D: Intracellular concentration of markers of cellular metabolism was observed in recovered/metabolism-activated Total Coliforms (A) and Ps. aeruginosa (B) cultures. Significantly low markers concentration was observed in metabolically shocked Coliforms (C) and A. hydrophilia treated by unspecific activators.

FIGS. 4A-C: Results of Coliform bacteria (A), Ps. aeruginosa (B) and Total bacteria (C) counts were plotted and correlating equations obtained for each contaminated type. Calculation models for initial contamination (Ni) were standardized for 1 ml of initial microbial suspensions.

FIG. 5: Media testing results correlation

FIG. 6: Media containing growth activators enables commencing of bacterial detection 20-80% sooner than standard growth media.

FIG. 7 Illustration of a system for rapid detection and quantification of specific live and recovered microorganisms in a sample.

### DETAILED DESCRIPTION OF PREFERRED EMBODIMENTS

### General

The current invention discloses a method for fast detection and identifying of recoverable microorganisms using combined microscopic detection of enhanced microbial metabolism by measuring of intracellular concentration of metabolism marker molecules as well as labeled non metabolic specific markers, preceded by enhanced microorganism growth using host-driven tissues or factors.

### Recovery of Stressed Microorganisms

Microorganisms present in processed food, water or the environment may be injured and hence more exacting in their growth requirements. Such organisms may be difficult to detect because they fail to grow on the selective media normally used in their isolation. Failing in bacteria isolation is also a known phenomenon in a significant number of infections in human patients. Infections without an isolated causative agent are particularly frequent in some human body districts. Furthermore, a significant number of bacterial infection cases are not associated with the isolation of the causative agent when standard microbiological methods, based on isolation of bacteria in culture media, are applied. This inadequate bacterial isolation might be due to the involvement of microorganisms hardly growing or non-recoverable in culture media.

An appreciation of the nature of sub-lethal injury and its repair by recovering microorganisms, is therefore important in detecting and enumerating microbes.

### Recoverable Microorganisms:

Viable microorganisms include forms of non-recoverable and recoverable microorganisms. Recoverable microorganisms are of the highest interest and most tested in conventional microbial tests of water, food, beverages, medicine etc. To date, the only certified identifying and quantification method for this type of the tests is conventional count of Colony forming units (CFU).

The current invention is based on the current finding that metabolism rate is much higher in recoverable microorganisms when recovering and that metabolic processes are activated by inducers specific for microbial type. More specifically, metabolism's marker compounds' dyes increase their concentration in recoverable cells with increase of metabolism.

The method disclosed by current invention is usable to practically detect any recoverable microorganism by microscopic detection of specifically activated metabolism, without the need to wait for grown of colony (HPC method).

Based on this finding, current invention discloses an innovative technology to identify recoverable microorganisms in a short time. The technology combines identifying of the type of microbial cells by detecting metabolism rate, once said metabolism rate has been increased by specific inducers (metabolic activators). The metabolism rate and the intracellular concentration level of marker compounds are a function of cell activity, which is very high in recovered cells.

Additionally, quantification of intracellular concentration of marker molecules is usable to differentiate recovered (very active) from inactive ones.

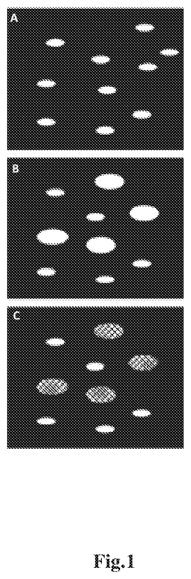

Using the specific dye for the marker molecules it is possible to detect and quantify the rate and level of intracellular concentration of said molecules, in order to define, specify and count recovered microorganisms in tested sample as shown in FIG. 1.

### Coliform Bacteria

Coliform bacteria refer hereinafter to rod-shaped Gram-negative non-spore forming and motile or non-motile bacteria which ferment lactose with the production of acid and gas when incubated at 35-37° C. They are a commonly used indicator of sanitary quality of foods and water. Coliforms are found in the aquatic environment, in soil and on vegetation; they are universally present in large numbers in the feces of warm-blooded animals. While coliforms themselves are not normally causes of serious illness, their presence is used to indicate that other pathogenic organisms of fecal origin may be present. Such pathogens include disease-causing bacteria, viruses, or protozoa and many multicellular parasites.

### CFU

Colony forming units, usually abbreviated as CFU, refer hereinafter to individual colonies of bacteria, yeast or mold. A colony of bacteria or yeast refers to a mass of individual cells of same organism, growing together. For molds, a colony is a group of hyphae (filaments) of the same mold growing together. Colony forming units are used as a measure of the number of microorganisms present in or on surface of a sample. Colony forming units may be reported as CFU per unit weight, CFU per unit area, or CFU per unit volume depending on the type of sample tested. To determine the number of colony forming units, a sample is prepared and spread or poured uniformly on a surface of an agar plate and then incubated at some suitable temperature for a number of days. The colonies that form are counted. CFU is not a measure for individual cells or spores as a colony may be formed from a single or a mass of cells or spores.

### HPC—Heterotrophic Plate Count

The heterotrophic plate count (HPC), formerly known as the standard plate count, is a procedure for estimating the number of live heterotrophic bacteria in water. This test provides useful information about water quality and supporting data on the significance of coliform test results.

The HPC is useful, inter alia, in judging the efficiency of various treatment processes for drinking water, swimming pools, as well as for checking the quality of finished water in a distribution system. Also for applications where boilers and cooling towers are present. Heterotrophs refer hereinafter to microorganisms that require organic carbon for growth. These include bacteria, yeasts and molds. A variety of simple culture-based tests that are intended to recover a wide range of microorganisms from water are collectively referred to “heterotrophic plate count” or “HPC test” procedures.

The current invention discloses a methodology to identify and quantify recoverable microorganisms in a short time, based on microscopic detection of cellular metabolism within said microorganisms.

More detailed, the current invention discloses a method for a rapid and high-yield detection of a specific microorganism in a sample, comprising mixing the sample with a unique detection cocktail, and determining the number of specific microorganisms in said sample by measuring gray levels of fluorescently marked intracellular indicators of microbial metabolism and fluorescently conjugated specific non-metabolic marker molecules in said mixture; said gray levels correlating with quantity of recovered high metabolic-active said microorganism in said sample.

Said cocktail comprises: (i) a nutrient medium comprising host-derived fraction containing cell growth activators from said host organism, for accelerated selective growing and multiplying of said microorganism; (ii) a fluorescently-labelled specific markers of the tested microbe specific for recognizing said microorganism; and (iii) a metabolic activator for increasing intra-cellular metabolism and at least one a fluorescently detectable marker factor that can be indicator of the said metabolism. Reference is now made to an embodiment of the present invention disclosing a method for rapid detection and quantification of specific live and recovered microorganisms in a sample, comprising steps of:
a. providing a detection cocktail comprising
   i. a nutrient medium comprising host-derived fraction containing cell growth activators from said host organism, for accelerated selective growing and multiplying of said microorganism;
   ii. at least one fluorescent metabolism marker molecule detectable by a sensor,
   iii. at least one metabolic activator for specifically increasing metabolism of said specific microorganism and increasing of intracellular concentration of said fluorescent marker molecule into said specific microorganism;
b. contacting said sample with said detection cocktail;
   wherein said detection and quantification is by steps of
   iv. measuring gray levels of fluorescent intensity of said marker molecules by said sensor, said gray levels correlated to at least one of intracellular metabolism of recoverable or recovered said specific microorganism,
   v. determining said gray level to a predetermined threshold of metabolism;
   further correlating said gray levels above said predetermined threshold with quantity of recovered high metabolic-active said microorganism in said sample.

Reference is now made to an embodiment of the present invention disclosing the method mentioned above, wherein said detection cocktail further comprises a fluorescently labelled specific microbial marker configured to specifically recognizing said microorganisms.

Reference is now made to an embodiment of the present invention disclosing the method mentioned above, wherein said microorganism selected of bacteria, fungi, viruses, protozoa, yeasts, molds, parasites and any combination thereof.

Reference is now made to an embodiment of the present invention disclosing the method mentioned above, wherein said sample comprising at least one of fluid, water, food, beverage, blood, a solution, a pharmaceutical preparation, a mammalian tissue, air, soil, surface, and any combination thereof.

Reference is now made to an embodiment of the present invention disclosing the method mentioned above, wherein host-derived fraction comprises host tissue, host tissue extract, host growth factor, and any combination thereof.

Reference is now made to an embodiment of the present invention disclosing the method mentioned above, wherein host tissue is selected from a group comprising somatic tissue, neuronal tissue, digestive tract tissue, skin, epithelial tissue, connective tissue, muscular tissue, adipose tissue, areolar tissue, bone tissue, cartilage tissue, lymphatic tissue, muscular tissue, fibrous tissue, urinary tract tissue, lymphatic tissue, liver tissue, blood serum, fetal blood serum, cerebrospinal fluid, urine, saliva, sweat, breast milk, and any combination thereof.

Reference is now made to an embodiment of the present invention disclosing the method mentioned above, wherein said metabolic activator is selected from a group comprising sugars, proteins, effectors of membrane receptors, substrates of intercellular enzymatic reactions and any combination thereof.

Reference is now made to an embodiment of the present invention disclosing the method mentioned above, wherein said marker molecule is selected from a group consisting conjugated antibodies, metabolized carbonates, DNA or RNA sequences, products of intracellular reactions, cell membrane parts, membrane receptors, specific effectors, extracellular liquids and any combination thereof.

Reference is now made to an embodiment of the present invention disclosing the method mentioned above, wherein metabolic activators are Asparagine for Ps. aeruginosa, Beta-galactoside for Total Coliform bacteria and NB medium for Total bacteria count.

Reference is now made to an embodiment of the present invention disclosing the method mentioned above, wherein said labelled antibody is selected from the group consisting of specific microbial markers with ferromagnetic moieties, antibodies with paramagnetic moieties, antibodies with diamagnetic moieties, antibodies with fluorescent moieties, antibodies with phosphorescent moieties, antibodies with luminescent moieties, antibodies with electro-chemiluminescent moieties, chromatic moieties, antibodies with moieties having a detectable electron spin resonance, antibodies with electrical capacitance, antibodies with dielectric constant or antibodies with electrical conductivity, and any combination thereof.

Reference is now made to an embodiment of the present invention disclosing the method mentioned above, wherein antibody complex comprises antibody and antigen, further wherein antigens are selected from a group of amino acids, peptides, sugars, monosaccharides, polysaccharides, lipids and any combination thereof.

Reference is now made to an embodiment of the present invention disclosing the method mentioned above, wherein said filtering further comprising a step of filtering via bacterial filter of arrange of 0.2 to 0.6 μm pore.

Reference is now made to an embodiment of the present invention disclosing the method mentioned above, wherein time period for said detection of a specific organism in a sample is less than 24 hours.

Reference is now made to an embodiment of the present invention disclosing the method mentioned above, wherein said method further comprising steps of increasing the number of recovered microorganism is said sample.

Reference is now made to an embodiment of the present invention disclosing the method mentioned above, wherein said metabolic activator is one of, asparagine, beta-galactoside; glucuronoside or NB medium and any combination thereof.

Reference is now made to an embodiment of the present invention disclosing the method mentioned above, wherein number of said specific microorganism in said sample is equal or higher than the number of Colony Forming Units of said specific organism detected by conventional Hetero-Plate Count.

Reference is now made to an embodiment of the present invention disclosing the method mentioned above, wherein Cetrimide and Fucidin are metabolic activators for Ps. aeruginosa.

Reference is now made to an embodiment of the present invention disclosing the method mentioned above, wherein X-glucuronide or B-galactose or D-Glucose, and any combination thereof, are said selective activators for Coliform bacteria.

Reference is now made to an embodiment of the present invention disclosing the method mentioned above, wherein said marker molecules are selected of Fluorescein, Fluorescein di(β-D-glucuronide), NileRed, or CY5, and any combination thereof.

Reference is now made to an embodiment of the present invention disclosing the method mentioned above, wherein said antibody is selected of Anti-Pseudomonas aeruginosa monoclonal antibody and Anti-E. coli FITC.

Reference is now made to an embodiment of the present invention disclosing the method mentioned above, wherein said nutrient medium, BcS-EC growth medium, for selective said growing of Coli-forming bacteria comprises a mixture of Peptone water, McConkey Broth, DMEM, Fetal Calf Serum, 4-Nitrophenyl β-D-glucuronide, Galactose and glucose.

Reference is now made to an embodiment of the present invention disclosing the method mentioned above, for selective said growing of Listeria comprises a composition of Listeria Broth, DMEM, Fetal Calf Serum, Nalidixic acid, Cycloheximide, Galactose, Glucose and FBS.

Reference is now made to an embodiment of the present invention disclosing a detection cocktail for rapid detection of the number of live and recovered specific microorganism in a sample, comprising of:
a. a nutrient medium comprising host-derived fraction, for accelerated selective growing and multiplying of said microorganism;
b. at least one fluorescent metabolism marker molecule that can be detectable by a sensor
c. at least one metabolic activator for specifically increasing metabolism of said specific microorganism and increasing of intracellular concentration of said fluorescent marker molecule into said specific microorganism;
   wherein said detection cocktail, when in contact with a sample containing live predetermined microorganisms is a source of fluorescence detectable by a sensor sufficient to provide gray levels of said fluorescent marker molecules; said gray levels above predetermined threshold correlated with quantity of recovered high metabolic-active said microorganism in said sample.

Reference is now made to an embodiment of the present invention disclosing the method mentioned above, wherein said detection cocktail further comprises a fluorescently labelled antibody complex configured to bind to antigens specific for recognizing said microorganisms.

Reference is now made to an embodiment of the present invention disclosing the method mentioned above, wherein said microorganism selected of bacteria, fungi, viruses, protozoa, yeasts, molds, parasites and any combination thereof.

Reference is now made to an embodiment of the present invention disclosing the method mentioned above, wherein said sample comprises at least one of fluid, water, food, beverage, blood, a solution, a pharmaceutical preparation, a mammalian tissue, air, soil, surface, and any combination thereof.

Reference is now made to an embodiment of the present invention disclosing the method mentioned above, wherein host-derived fraction comprises host tissue, host tissue extract, host growth factor, and any combination thereof.

Reference is now made to an embodiment of the present invention disclosing the method mentioned above, wherein host tissue is selected from a group comprising somatic tissue, neuronal tissue, digestive tract tissue, skin, epithelial tissue, connective tissue, muscular tissue, adipose tissue, areolar tissue, bone tissue, cartilage tissue, lymphatic tissue, muscular tissue, fibrous tissue, urinary tract tissue, lymphatic tissue, liver tissue, blood serum, fetal blood serum, cerebrospinal fluid, urine, saliva, sweat, breast milk, and any combination thereof.

Reference is now made to an embodiment of the present invention disclosing the method mentioned above, wherein said metabolic activator is selected from a group comprising sugars, proteins, effectors of membrane receptors, substrates of intercellular enzymatic reactions and any combination thereof.

Reference is now made to an embodiment of the present invention disclosing the method mentioned above, wherein said marker molecule is selected from a group consisting conjugated antibodies, metabolized carbonates, DNA or RNA sequences, products of intracellular reactions, cell membrane parts, membrane receptors, specific effectors, extracellular liquids and any combination thereof.

Reference is now made to an embodiment of the present invention disclosing the method mentioned above, wherein metabolic activators are Asparagine for Ps. aeruginosa, Beta-galactoside for Total Coliform bacteria and NB medium for Total bacteria count.

Reference is now made to an embodiment of the present invention disclosing the method mentioned above, wherein said labelled antibody is selected from the group consisting of antibodies with ferromagnetic moieties, antibodies with paramagnetic moieties, antibodies with diamagnetic moieties, antibodies with fluorescent moieties, antibodies with phosphorescent moieties, antibodies with luminescent moieties, antibodies with electro-chemiluminescent moieties, chromatic moieties, antibodies with moieties having a detectable electron spin resonance, antibodies with electrical capacitance, antibodies with dielectric constant or antibodies with electrical conductivity, and any combination thereof.

Reference is now made to an embodiment of the present invention disclosing the method mentioned above, wherein antibody complex comprises antibody and antigen, further wherein antigens are selected from a group of amino acids, peptides, sugars, monosaccharides, polysaccharides, lipids and any combination thereof.

Reference is now made to an embodiment of the present invention disclosing the method mentioned above, wherein said filtered mixture is via a bacterial filter of a range of 0.2 to 0.6 um pore.

Reference is now made to an embodiment of the present invention disclosing the method mentioned above, wherein time period for said detection of a specific organism in a sample is less than 24 hours.

Reference is now made to an embodiment of the present invention disclosing the method mentioned above, wherein said composition further suitable to increase the number of recovered said microorganisms in said sample.

Reference is now made to an embodiment of the present invention disclosing the method mentioned above, wherein a metabolic activator for increased intra-cellular internalization additionally comprises at least one of CY3, Asparagine, beta-Galactoside or NB medium and any combination thereof.

Reference is now made to an embodiment of the present invention disclosing the method mentioned above, wherein number of said specific microorganism in said sample is equal or higher than the number of Colony Forming Units of said specific organism detected by conventional Hetero-Plate Count.

Reference is now made to an embodiment of the present invention disclosing the method mentioned above, wherein said Cetrimide and Fucidin are said metabolic activators for Ps. aeruginosa.

Reference is now made to an embodiment of the present invention disclosing the method mentioned above, wherein X-glucuronide or B-galactose or D-Glucose, and any combination thereof, are said selective activators for Coliform bacteria.

Reference is now made to an embodiment of the present invention disclosing the method mentioned above, wherein said marker molecules are selected of Fluorescein, Fluorescein di(β-D-glucuronide), NileRed, or CY5, and any combination thereof.

Reference is now made to an embodiment of the present invention disclosing the method mentioned above, wherein said antibody is selected of Anti-Pseudomonas aeruginosa monoclonal antibody and Anti-E. coli FITC.

Reference is now made to an embodiment of the present invention disclosing the method mentioned above, wherein said nutrient medium, BcS-EC growth medium, for selective said growing of Coli-forming bacteria comprises a mixture of Peptone water, McConkey Broth, DMEM, Fetal Calf Serum, 4-Nitrophenyl β-D-glucuronide, Galactose and glucose.

Reference is now made to an embodiment of the present invention disclosing the method mentioned above, for selective said growing of Listeria, comprises a composition of Listeria Broth, DMEM, Fetal Calf Serum, Nalidixic acid, Cycloheximide, Galactose, Glucose and FBS.

Reference is now made to an embodiment of the present invention disclosing a system useful for rapid detection and quantification of specific live and recovered microorganisms in a sample, said system comprises of:
a. an in vitro contact module configured to contact said sample with a detection cocktail, said cocktail comprises:
   i. a nutrient medium comprising host-derived fraction containing cell growth factors, for accelerated selective growing and multiplying of said microorganism;
   ii. at least one fluorescent metabolism marker molecule that can be detectable by a sensor
   iii. at least one metabolic activator for specifically increasing metabolism of said specific microorganism and increasing of intracellular concentration of said fluorescent marker molecule into said specific microorganism;
b. a module for recording data on the outcome of said in vitro contacting wherein said module comprises:
   i. a sensor for measuring gray levels of fluorescent intensity of said marker molecules; and
   ii. a module for correlating said detected gray levels to metabolism of said specific microorganism, determining said gray level to a predetermined threshold of metabolism; and further correlating said gray levels above said predetermined threshold with quantity of recovered high metabolic-active said microorganism in said sample.

### EXAMPLES

The following samples describe the methods and apparatus used, and summarize the results of the experimental work for developing a recoverable microorganism quantification method based on detection of the rate of the cellular metabolism in specifically recovered microbes, compared to the metabolism in non-activated microbes. In addition, the document describes process of testing system design and validation.

### Example 1

### Methodology for Fast Identifying and Quantification of Recoverable Microorganisms Using Microscopic Detection of Cellular Metabolism

Viable microorganisms include forms of non-recoverable and recoverable microorganisms. Recoverable microorganisms are of the highest interest and most tested in conventional microbial tests of water, food, beverages, medicine etc. To date, the only certified identifying and quantification method for this type of the tests is conventional count of Colony forming units (CFU). Microbial recovery phenomenon occurs in heterotrophic plate count (HPC) testing, when microbes reproduce multiply using media containing recovery/growth activating specific and general nutrients.

Increased metabolism phenomenon is characteristic of active cells only and increases according to cell metabolism level. Quantification of intracellular concentration of marker molecules is used to differentiate recovered (very active) from inactive ones. As part of increased metabolism, cell membrane parts, membrane receptors, specific effectors and extracellular liquids are internalized and concentrate inside the cell. The level of intracellular concentration of indicators of intracellular metabolism is a function of cell activity, which is very high in recovered cells.

Using the marker molecules' specific dye it is possible to detect and quantify the rate and level of the said indicators intracellular concentration, in order to define, specify and count recovered microorganisms in tested sample as shown in the diagram below (as presented in FIG. 1).

FIG. 1. presents intracellular concentrating of marker compound in recoverable microorganisms. FIG. 1 further presents the effect of bacterial recovery or metabolism stimulators/activators on concentrations of dyes of marker molecules in various cells. Dyes of marker molecules increase their concentration in recoverable cells with increase metabolic rate. The intracellular concentration of said marker molecules is low in non-recoverable cells: In non-growth and anabiotic microorganisms which have low level of metabolism, the dyes remain confined to the extracellular space.

### Methodology Development

The proposed invention comprises a method based on detection of cellular metabolic rate to determine the type and number of recovered microbes in a media. The detection results of the proposed invention were further to those obtained with conventional CFU methods further revealed an equivalence between those methods, allowing the proposed invention to be used as a CFU counter.

The method was developed and validated to use for analysis of Total microbial count, Total Coliform bacteria and Ps. aeruginosa in different testing media. All tests were performed using standard microscopic, imaging and conventional microbiology lab equipment.

The main principles of methodology development are as following:
a. Determination of nutrients cocktail composition for optimal recovery of tested microbial type that contains among other things cell growth factors from host tissue
b. Determination of effectors which specifically stimulate cell metabolism in tested microbes
c. Determination of fluorescent marker molecules for intracellular metabolism in the said microbial cells
d. Development of method for preparing the tested media for microscopic examination
e. Determination of incubation conditions (minimal incubation time and temperature) for tested media in nutrient cocktail in order to allow microbes' recovery and microscopic examination in required resolution
f. Determination of conditions (time) for cell sample staining with labeled marker molecules in presence of the metabolism effectors/activators.
g. Determination of conditions for microscopic examination
h. Determination image definition for tested recovered microbes
i. Plotting of functions for correlation between microscopic cell counts and HPC counts regarding to specific testing application

### Samples:

Samples were collected of:
a. Sludge from municipal wastewater treatment plant
b. Pasteurized milk (3%) fat
c. Freshly squeezed orange juice.

Sampling was performed at three different days or of three production batches in triplicates from each source (total of 27 samples for each testing media). Wastewater sludge was tested for total Coliform bacterial, pasteurized milk for Ps. aeruginosa and orange juice for Total bacterial contamination.

The specific recovered bacteria count was carried out in these samples. These samples were tested in parallel by conventional HPC methods relevant for the tested bacteria.

Data from all analyses was used to determine the relationship between the proposed invention's procedures for cell metabolism-based recoverable microbe's determination to the number of recovered microbes of HPC methods in tested media. The correlation analysis revealed a value of (R²)=0.95 comparing proposed invention to the conventional method.

### Tested Media Preparation

Wastewater sludge was diluted by iso-normal PBS in concentrations 1:10, 1:100 and 1:1000 w/v. One ml of each solution was added to 9 ml of standard MacConkey Broth (Mb) growing media and incubated using shaking incubator et 35° C. for 1 hr. Within incubation the solutions were filtered via 2.0μ and 0.1 ml of the filtrate was transferred via 0.4 μm membranes.

Pasteurized milk was contaminated with calibrated Ps. aeruginosa culture in concentrations of 1, 10 and 100 CFU/ml. Then, 10 ml of each contaminated milk samples were added to 90 ml of cocktail contained Pseudomonas Selective Broth (CB) and Tween 40 (2% v/v) and incubated et 25° C. for 7 hr. Within incubation 10 ml of the solutions were treated with HCl (pH 4.0) for 5 min, filtered via 2.0μ and 0.1 ml of the filtrate was transferred via 0.4 μm membranes. The membranes were washed additionally by iso-normal PBS.

Orange juice was contaminated with calibrated Total bacterial culture mix in concentrations of 1, 10 and 100 CFU/ml. Then 10 ml of each contaminated juice samples were added to 90 ml of NB and incubated using shaking incubator et 25° C. for 8 hr. Within incubation the solutions were filtered via 2.0μ and 0.1 ml of the filtrate was transferred via 0.4 μm membranes.

### Recovered Bacteria Staining and Imaging:

Following preparations, 0.4 μm microbial membranes were treated by working solutions of CY3 membrane dyes in PBS with additions of cell metabolic activators:
a. Asparagine for Ps. aeruginosa
b. Beta-galactoside for Total Coliform bacteria
c. NB medium for Total bacteria count
   in standard concentrations. Each membrane was stained at room temperature for 6 min., washed by PBS and imaged.

Imaging was done using an Axioplan 100 microscope, 0.5 NA Plan Neofluor X20 objective (BP 450-490 excitation filter (Excitation: 450-490 nm; Beam splitter: FT 510 nm; Emission: 515-555 nm; Karl Zeiss). Preparations were imaged by standard Axiocam 506 mono 5 megapixel CCD. Data collection, image processing and analyzing were performed by ImagePro+software.

### Recoverable Bacteria Microscopic Characteristics Finding:

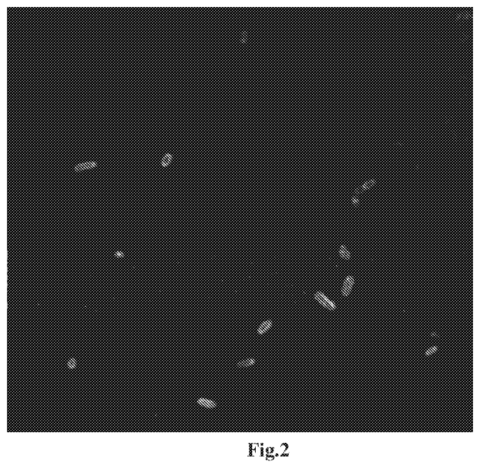

a. Each type of the tested bacteria were prepared by two different procedures:
b. Following proliferation bacterial suspension were dispensed for two same portions. The first portion was proliferated by our procedures as described previously. The second portion was shocked metabolically by incubation in iso-normal saline at 25° C. for 72 hr. The first portion was then stained in presence and the second portion in the absence of cell metabolic activators (see previous paragraphs).
c. The recovery activated and shocked cultures were microscopically observed and imaged.
d. Morphology and light intensity characteristics of microbes in each culture were measured and analyzed. Morphology characteristics of bacteria were similar in activated and shocked cultures regarding to specific bacterial type (FIG. 2).
e. Along with this, fluorescent intensity of activated bacteria was found to be significantly higher.

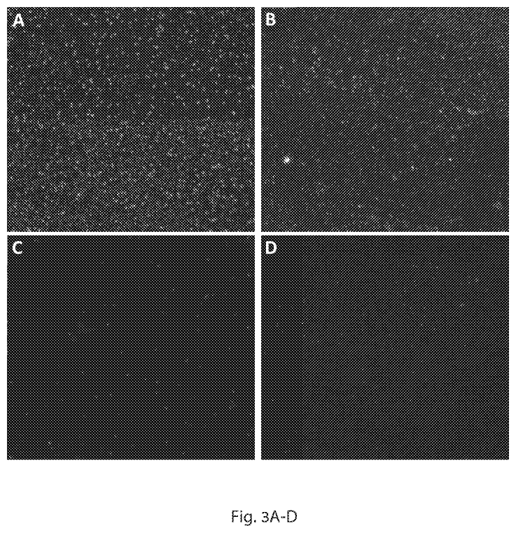

The following observations were made during imaging by current imaging setup:
a. The fluorescent intensity of stained bacteria was in the range of 25-225 gray levels (on a scale of 0 to 255)
b. Shocked bacteria had a signal of 25-55 gray levels
c. Cellular metabolism-activated microorganisms had a signal of 85-225 gray levels (FIGS. 3A-D).

Therefore, a difference of about 30 gray levels was measured between the recovered (cellular metabolism-stimulated) and non-recovered microorganisms.

FIGS. 3A-D presents intracellular concentrating of marker molecules in recovered or metabolism-activated Total Coliforms (A) and Ps. aeruginosa (B) cultures. Significantly low concentration of marker molecules was observed in metabolically shocked Coliforms (C) and in A. hydrophilia treated by unspecific activators (D).

### Characteristics Used for Recovered Microbial Cells Identifying in Tested Media.

### Correlations of Proposed Invention to Conventional (HPC) Counts:

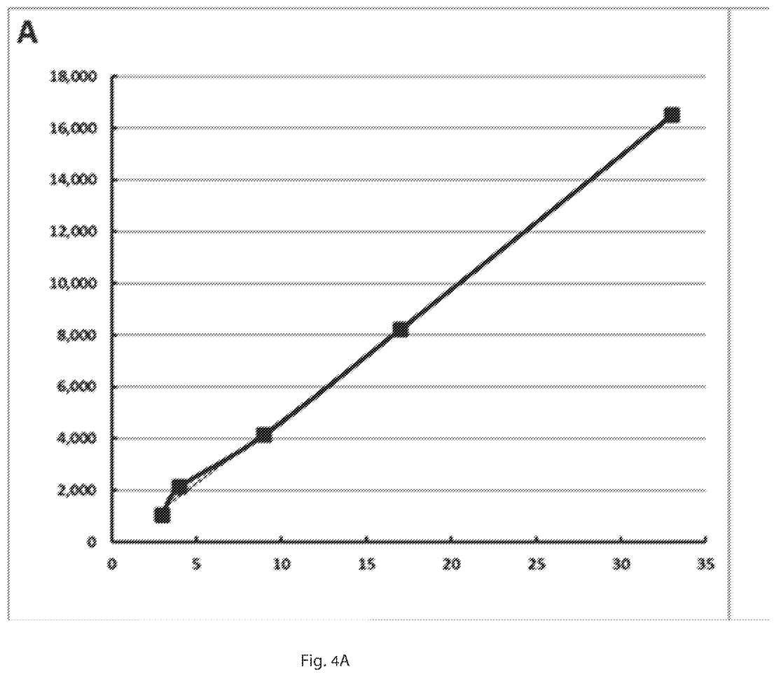

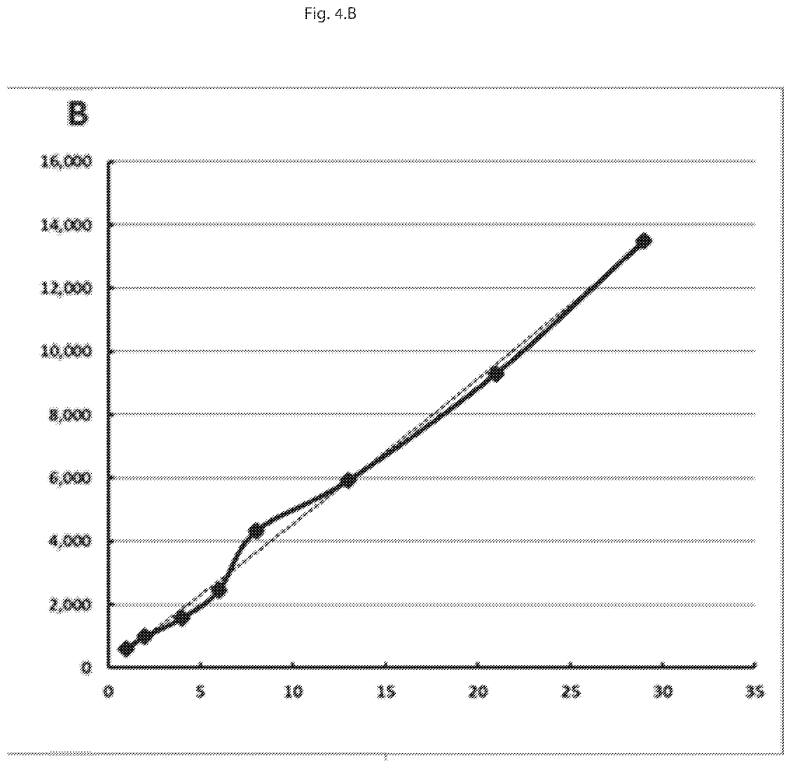

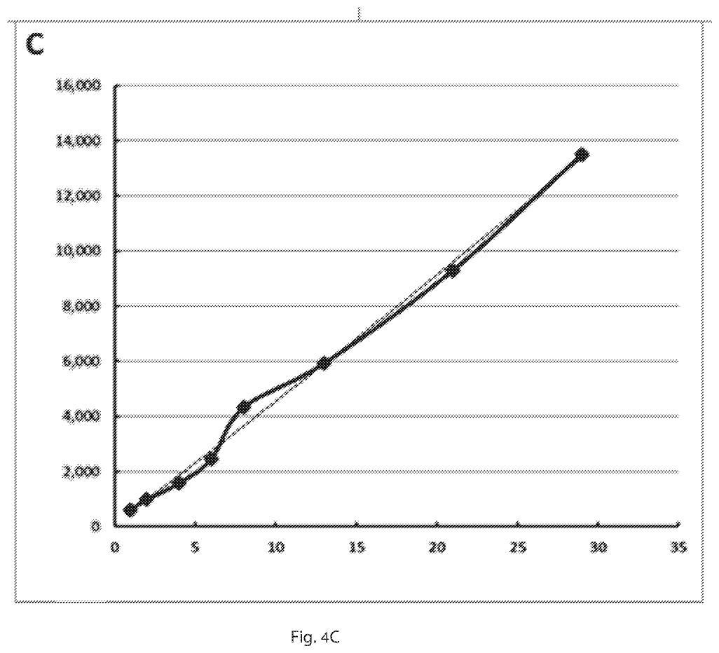

Samples of Total Coliform bacteria isolated from wastewater, Total bacteria mix obtained from orange juice and standard Ps. aeruginosa culture were diluted by PBS to concentrations of 1 and 10 CFU/ml. One ml of each diluted sample was added to selective bacteria recovery activating media and incubated as described previously. Then the solutions were treated, stained and imaged according to proposed invention, each for 0.5 hr. Twenty percent (20%) of each preparation was imaged. In parallel, 1 ml of each solution was tested using conventional HPC methodology. All tests were prepared in triplicates. The results obtained from both methods were averaged, normalized to 1 ml of starting microbial suspensions, compared (See table 1) and plotted. Correlation equations for each culture are shown in graphs (See FIGS. 4A-C).

FIG. 4A-C present the counting results of Coliform bacteria (A), Ps. aeruginosa (B) and Total bacteria (C). The counts values were obtained wither using the new method of proposed invention (x axis) or by conventional HPC methodology (y-axis). Counts were plotted and correlating equations were obtained for each contamination type. Calculation models for initial contamination (Ni) were standardized for 1 ml of initial microbial suspensions.

The initial contamination levels for tested media were calculated as following:

Ni=Fc(x)/2^{(Ts/Tc)}
Where: Ni—Initial contamination level;
   Fc—Correlation equation (experimental data);
   x—Count by New developed method [RMU];
   Ts—Sampling (incubation) time [min.];
   Tc—Cell cycle (dividing) period [min.] (experimental data).

TABLE 1
Microbial counts of New developed method (RMU-recovered microbial count)
correlated to HPC method (CFU-colony forming units). Results for Coliforming
bacteria (A), Ps. aeruginosa (B) and Total bacterial count (C) are
presented in form of raw and normalized to 1 ml of results.

| | Initiated contamination 1 CFU/ml | | | | Initiated contamination 10 CFU/ml | | | |
|---|---|---|---|---|---|---|---|---|
| | HPC count | | New method | | HPC count | | New method | |
| Sample | Count | Norm. | Count | Norm. | Count | Norm. | Count | Norm. |
| [hr.] | [CFU] | [CFU/ml] | [RMU] | [RMU/ml] | [CFU] | [CFU/ml] | [RMU] | [RMU/ml] |

A Coliforming bacteria

| | | | | | | | | |
|---|---|---|---|---|---|---|---|---|
| 0.0 | ND | NP | ND | NP | 0 | NP | ND | NP |
| 2.0 | 2 | 20 | ND | NP | 14 | 140 | ND | NP |
| 3.0 | 7 | 70 | ND | NP | 66 | 660 | ND | NP |
| 4.0 | 26 | 260 | ND | NP | 241 | 2,410 | 5 | 2,500 |
| 5.0 | 104 | 1,040 | 3 | 1,500 | 885 | 8,850 | 17 | 8,500 |
| 5.5 | 212 | 2,120 | 4 | 2,000 | 1,542 | 15,420 | 34 | 17,000 |
| 6.0 | 415 | 4,150 | 9 | 4,500 | TNTC | NP | 68 | 34,000 |
| 6.5 | 822 | 8,220 | 17 | 8,500 | TNTC | NP | 114 | 57,000 |
| 7.0 | 1,650 | 16,500 | 33 | 16,500 | TNTC | NP | 242 | 121,000 |
| 7.5 | TNTC | TNTC | 65 | 32,500 | TNTC | NP | 511 | 255,500 |
| 8.5 | TNTC | TNTC | 262 | 131,000 | TNTC | NP | 2,236 | 1,118,000 |
| 9.5 | TNTC | TNTC | 1,158 | 579,000 | TNTC | NP | TNTC | NP |
| 12.0 | TNTC | TNTC | TNTC | NP | TNTC | NP | TNTC | NP |

B Ps. aeruginosa

| | | | | | | | | |
|---|---|---|---|---|---|---|---|---|
| 0.0 | ND | NP | ND | NP | ND | NP | ND | NP |
| 3.0 | 2 | 20 | ND | NP | 15 | 150 | ND | NP |
| 4.0 | 3 | 30 | ND | NP | 36 | 360 | ND | NP |
| 6.0 | 24 | 240 | ND | NP | 246 | 2,460 | 5 | 2,560 |
| 7.0 | 61 | 610 | 1 | 500 | 621 | 6,210 | 13 | 6,451 |
| 7.5 | 99 | 990 | 2 | 1,000 | 884 | 8,840 | 22 | 11,000 |
| 8.0 | 158 | 1,580 | 4 | 2,000 | 1,488 | 14,880 | 33 | 16,255 |
| 8.5 | 245 | 2,450 | 6 | 3,000 | 2,287 | 22,870 | 58 | 29,000 |
| 9.0 | 432 | 4,320 | 8 | 4,000 | TNTC | NP | 82 | 40,960 |
| 9.5 | 593 | 5,930 | 13 | 6,500 | TNTC | NP | 141 | 70,500 |
| 10.0 | 930 | 9,300 | 21 | 10,500 | TNTC | NP | 206 | 103,213 |
| 10.5 | 1,350 | 13,500 | 29 | 14,500 | TNTC | NP | 344 | 172,000 |
| 12.0 | TNTC | NP | 128 | 64,000 | TNTC | NP | 1,311 | 655,360 |

C Total bacteria

| | | | | | | | | |
|---|---|---|---|---|---|---|---|---|
| 0.0 | ND | NP | ND | NP | ND | NP | ND | NP |
| 3.5 | 2 | 20 | ND | NP | 16 | 160 | ND | NP |
| 5.5 | 8 | 80 | ND | NP | 89 | 890 | 2 | 1,000 |
| 7.5 | 49 | 490 | ND | NP | 421 | 4,210 | 12 | 6,000 |
| 8.0 | 68 | 680 | 2 | 1,000 | 687 | 6,870 | 15 | 7,500 |
| 8.5 | 102 | 1,020 | 3 | 1,500 | 1,016 | 10,160 | 26 | 13,000 |
| 9.0 | 142 | 1,420 | 5 | 2,500 | 1652 | 16,520 | 38 | 19,000 |
| 9.5 | 231 | 2,310 | 7 | 3,500 | TNTC | NP | 49 | 24,500 |
| 10.0 | 378 | 3,780 | 8 | 4,000 | TNTC | NP | 92 | 46,000 |
| 10.5 | 544 | 5,440 | 14 | 7,000 | TNTC | NP | 142 | 71,000 |
| 11.0 | 870 | 8,700 | 22 | 11,000 | TNTC | NP | 201 | 100,500 |
| 11.5 | 1120 | 11,200 | 30 | 15,000 | TNTC | NP | 312 | 156,000 |
| 12.0 | TNTC | NP | 48 | 24,000 | TNTC | NP | 511 | 255,500 |

Comparison of Wild Diluted Samples with Contaminated Samples

Total microbial mix from orange juice was cultured in broth medium for 72 hr at 25° C. The medium was removed by centrifugation and microbial pellet was used for juice contamination of 1, 10 and 100 CFU/ml. Pasteurized milk was contaminated with standard Ps. aeruginosa by the same procedure. Wastewater sludge was taken as is and diluted as described previously.

Contaminated and wild diluted samples were tested in triplicates by conventional (HPC) and the new method of the proposed invention, simultaneously. The testing was repeated in three different experimental days for each kind of the test. Results of microscopic observations were processed for recovered bacteria counts as described previously. The counts of both kinds of tests were averaged regarding to experimental day, normalized for 1 ml of initial sample and correlated. Summary of the testing results is presented in table 2.

TABLE 2
Testing results of New developed method
(RMU) and HPC method (CFU).
A
Coliforming bacteria count in Wastewater sludge

| Experi- | | HPC count | New method | | |
|---|---|---|---|---|---|
| mental | Initial | Norm. | Raw | Norm. | Correlation |
| day | dillution | [CFU/gr] | [RMUl] | [RMU/gr] | [%] |
| 1 | 1:10 | 1,210 | 11 | 1,303 | 92 |
| | 1:100 | 1,390 | 13 | 1,552 | 88 |
| | 1:1000 | 1,320 | 12 | 1,427 | 92 |
| 2 | 1:10 | 2,040 | 17 | 2,053 | 99 |
| | 1:100 | 1,960 | 18 | 2,180 | 89 |
| | 1:1000 | 2,140 | 19 | 2,307 | 92 |
| 3 | 1:10 | 1,540 | 14 | 1,677 | 91 |
| | 1:100 | 1,520 | 14 | 1,677 | 90 |
| | 1:1000 | 1,730 | 15 | 1,802 | 96 |

B
Ps. aeruginosa bacteria count in Milk

| Experi- | Initial | HPC count | New method | | Corre- |
|---|---|---|---|---|---|
| mental | contamination | Norm. | Raw | Norm. | lation |
| day | [CFU/ml] | [CFU/ml] | [RMUl] | [RMU/ml] | [%] |
| 1 | 1 | ND | 3 | 2 | NC |
| | 10 | 8 | 13 | 9 | 88 |
| | 100 | 97 | 132 | 109 | 87 |
| 2 | 1 | 2 | 4 | 3 | 55 |
| | 10 | 26 | 42 | 31 | 82 |
| | 100 | 165 | 195 | 175 | 94 |
| 3 | 1 | 3 | 5 | 4 | 81 |
| | 10 | 31 | 48 | 35 | 86 |
| | 100 | 186 | 211 | 194 | 96 |

C
Total bacteria count in Orange iuice

| Experi- | Initial | HPC count | New method | | Corre- |
|---|---|---|---|---|---|
| mental | contamination | Norm. | Raw | Norm. | lation |
| day | [CFU/ml] | [CFU/ml] | [RMUl] | [RMU/ml] | [%] |
| 1 | 1 | 2 | 5 | 2 | 100 |
| | 10 | 12 | 19 | 13 | 92 |
| | 100 | 99 | 129 | 106 | 93 |
| 2 | 1 | 1 | 2 | 2 | 47 |
| | 10 | 8 | 14 | 10 | 75 |
| | 100 | 88 | 115 | 93 | 94 |
| 3 | 1 | ND | 1 | 1 | NC |
| | 10 | 9 | 14 | 10 | 90 |
| | 100 | 87 | 111 | 89 | 97 |

Coliforming bacteria (A) count in wastewater sludge, Ps. aeruginosa count in milk (B) and Total bacterial count in orange juice (C) are presented and normalized to 1 ml of results. The correlations per each test was calculated regarding to normalized results.

The results were obtained:
Conventional method: 24 hr. for Coliform bacteria
72 hr. for Ps. aeruginosa
96 hr. for Total bacterial count
   New method: 1 hr. for Coliform bacteria
7 hr. for Ps. aeruginosa
8 hr. for Total bacterial count

The testing time needed for results obtaining by the new method is depended and inversely proportional to starting contamination of tested media and microbial cell cycle (dividing time).

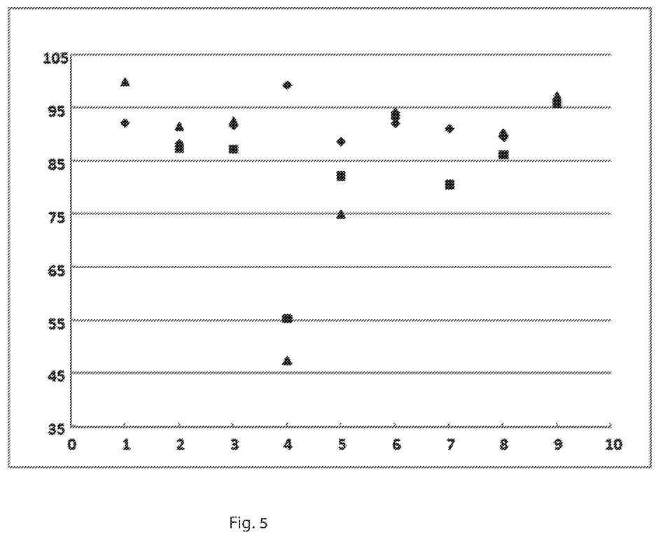

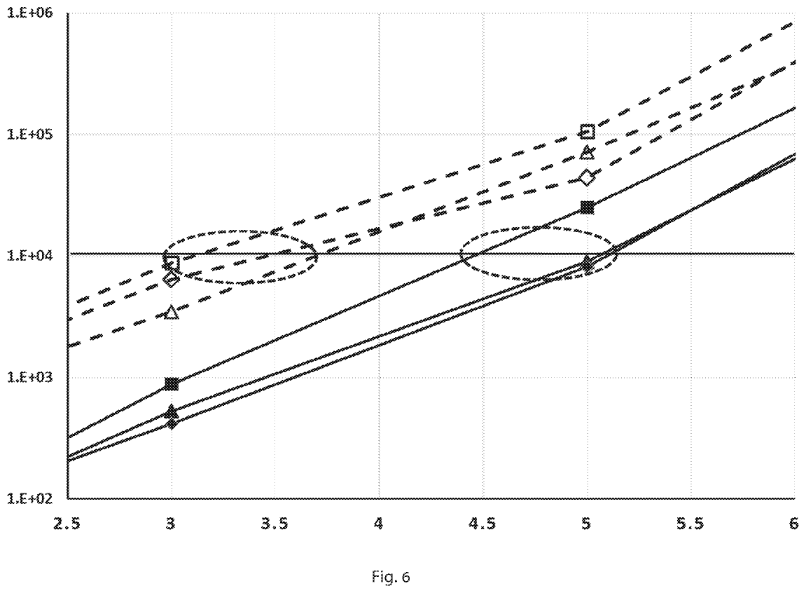

In most cases the correlation between the new and conventional method is defined as about 90% (See FIG. 5) which presents media testing correlation. FIG. 5 presents correlation between the tested methods and the sample number).

### Summary of Example 1

The results of example 1 show the validity of the new methodology of the proposed invention, and the equivalence of said new methodology to CFU counting method, for example samples of Total, Cohform and Ps. aeruginosa recoverable bacteria count in wastewater sludge, juices and milk.

Those three types' bacterial contaminations are of utmost importance in food, beverages and environmental microbiology.

Recoverable count using the new methodology will usually provide larger numbers than CFU tested conventionally: Conventional HPC count is an indirect test based on culture of microbes. The cultivability of a microorganism depends on properties of growing media and incubation conditions.

According to this, it is expected that direct new testing system detect more recovered cells.

Comparisons of conventional HPC and new methodology for recoverable microorganisms quantification

Characteristics of both testing methods are summarized below

TABLE 3
Comparison characteristics of conventional
method to current invention

| Criteria | New method | | Conventional HPC | |
|---|---|---|---|---|
| Working time (sampling and | 0.2-0.5 | hr. | 10 | min. |
| transportation not included) | | | | |
| Time to receive the results | 1-8 | hr. | 24 hr.-21 days | |
| Authorized personnel | Yes | | No | |
| required | | | | |
| Cost per test for performer | 1.5-2.0 $ | | 1.0 $ | |
| Cost per test for customer | 20-60 $ | | 8 $ | |
| Required equipment cost | 30-40k$ | | 25-35k$ | |
| Required working space | 25 | m³ | 1.5 | m³ |
| (min.) | | | | |
| Accuracy | ±35% | | ±15% | |
| Ability of total automation | No | | Yes | |
| Sensitivity | Low | | High | |
| Time required for periodical | 3 | months | 3 | months |
| validation | | | | |
| Time required for new | 3 | month | 2 | week |
| application validation | | | | |

| | | |
|---|---|---|
| Special working space | Yes | Yes |
| required | | |
| Ability to proceed close to | No | Yes |
| sampling or production | | |
| area | | |

### Example 2

### Preparation of Microbial Metabolism Activation/Staining Cocktail

The staining cocktail contains three main components. These components are used at the same time or separately, depending on the tested bacteria and/or tested material.

Specific or Non-Specific Activation of the Cellular Metabolism:

This reagent contains a substance that in selective or non-selective manner increase intracellular metabolism of the tested microbe. The substances that are usable are specific or no-specific sugars, proteins, effectors of membrane receptors, specific substrates of intracellular enzymatic reactions.

This reagent contains

### Detection of High Metabolism:

This reagent contains a fluorescent detectable substance that is absorbed by a microbial cell and accumulates in it due to the rise in cellular metabolism. Substances that participate in intracellular metabolic reactions that have light/fluorescent properties are suitable.

### Specific Identification of Microbes:

This reagent contains a fluorescent detectable substance that specifically and accurately identify type of microbial species.

The reagents are chosen of:
fluorescent conjugated antibodies
metabolized carbonates
specific DNA/RNA sequences
fluorescent products of specific intracellular reactions
   Note—In some cases, the use of the same substances meet both purposes.

Using specific dyes, it is possible to detect and quantify the rate and level of internalization of the selected compounds to count and identify recoverable microorganisms in a tested sample.

The following are non-limiting examples for cocktails preparations.
a. A cocktail for Ps. aeruginosa testing:
b. Bactosense Mediums for Pseudomonas Enhancement Growth.
c. Cetrimide (10 mg/l)-a metabolism activator.
d. Fucidin (10 mg/l)-a metabolism activator.
e. Fluorescein, a selective marker of metabolism naturally produced in metabolically active Ps. aeruginosa cells.
f. Anti-Pseudomonas aeruginosa monoclonal antibody (for example ab35835) (1:50), an identification marker.
2. A cocktail for E. coli testing:
a. Bactosense Mediums for Coliform bacteria Enhancement grows
b. X-glucuronide (0.075 g/l), a metabolism activator.
c. B-galactose (1.0 g/l), a metabolism activator.
d. Fluorescein di(β-D-glucuronide) (5 mg/l), a selective metabolism marker.
e. Anti-E. coli FITC—(for example AB30522) (1:50), an identification marker.
3. A cocktail for Coliform bacteria testing:
a. Bactosense Mediums for Coliform bacteria Enhancement grows
b. D-Glucose (2 g/l), a metabolism activator.
c. B-galactose (1.0 g/l), a metabolism activator.
d. NileRed (5 mg/1), a general marker of metabolism.
   Or
e. CY5 (1 mg/1), a general marker of metabolism.

### Example 3

### Special Media for Enhancement Growth of Different Microorganisms

1. Growth media functions:
   a. Simulates host tissue/fluid environment.
   b. Includes various growth factors/stimulators/triggers.
   c. Controlled environmental parameters.
   d. Supports enhanced growth rates of microorganisms.
   a. Simulates host tissue/fluid environment

The current invention teaches that growth of pathogenic bacteria is stimulated by growth factors found in the host organism/tissue rather than those found in alternative sources. This is a logical assumption since a given pathogenic microbe will proliferate intensively in a specific type host organism/tissues, but will be harmless and/or will not proliferate in others.

Thus, it is possible to accelerate the microbial pathogen cultivation by addition of individual or mixed cell growth accelerators, which simulate host tissue/fluid environment, into the standard growth media.

This assumption was tested experimentally. The results indeed show acceleration of bacterial growth rate in ranges of 20-75%
b. Includes various growth factors/stimulators/triggers

The activator is chosen of:
i. Mixed/complicated activators:
   Tissues and/or tissue extracts from host of the pathogen—antibody free blood serum, fetal blood serum, somatic/neuronal tissue extracts, CSF, urine and/urine tract tissue extracts, lysozyme free saliva, etc. Mix of cell growth factors (cytokines) from the host organisms
ii. Isolated cytokines from the host organism/tissues.

For designing the growth media formulation, types and concentrations of the additions should be tested according to their influence on cultivation rate of the specific pathogen.
c. Design of formulation of the growth media:
i. Select the optimal standard selection medium (usually liquid) for the specific pathogen's growth.
ii. Select growth activator known to be strongly associated with the microorganism in the host.
iii. Select the environment that effectively supports growth of the host cells/tissues (standard mixture).
iv. Define concentration of the activator/s in the environment (activating mixture).
v. Define proportions between the standard and the activation mixtures in final formulation of the medium.
vi. Bring the content of selecting factors from the standard media up to the optimal levels.
d. Media quality test:
i. Compare microbial growth rates using Standard and Novell designed media by HPC or Five tubes methods.
ii. Define storage conditions and shelf life of the media by determining the microbial growth rate in it.
2. Examples for growth media
a. BcS-EC growth medium BcS-EC is a special bacteria growth media for accelerated specific growth of Coli-forming bacteria.

### Reagents:

For preparing of 1 L and/or 0.5 L BcS-EC media needed the following reagents are described in the table 4:

TABLE 4
Composition of BcS-EC media

| | | | Amount | | | | | |
|---|---|---|---|---|---|---|---|---|
| [symbol] o | Reagent | Storage | 500 ml | | 1000 ml | | Cat. number | Manufacturer |
| 1 | Peptone Water | RT | 125 | ml | 250 | ml | 7365A | Neogen |
| 2 | McConkey Broth | RT | 250 | ml | 500 | ml | 8468.0 0500 | VWR |
| | | | | | | | | Chemicals |
| 3 | DMEM (4.5 g)* | +4° C. | 100 | ml | 200 | ml | 11-055-1g | Biological Ind. |
| 4 | Fetal Calf Serum (FCS)* | −20° C. | 25 | ml | 50 | ml | 04-001-1A | Biological Ind. |
| 5 | 4-Nitrophenyl β-D- | RT | 10 | mg | 5 | mg | N1627 | Sigma |
| | glucuronide | | | | | | | |
| 6 | Galactose | +4° C. | 2.5 | g | 5 | g | G0625-100G | Sigma |
| 7 | Glucose | +4° C. | 1 | g | 2 | g | D8270-100G | Sigma |

*Unique additions for bacterial growth

### Procedure:

i. Prepare Peptone Water, McConkey Broth and DMEM according to the manufacturer's instructions:
ii. Dissolve 13.38 g DMEM High Glucose (4.5 g/L) in 1 L DDW with addition of 3.7 g/L Sodium Bicarbonate (without heating);
iii. Suspend 40.01 g MacConkey Broth in 1 L DDW with magnet stirrer and heating;
iv. Dissolve 15 g Peptone Water in 1 L DDW with magnet stirrer and heating;
v. Mix together Peptone Water, MacConkey and DMEM solutions.
vi. Add FCS, Galactose and Glucose to this solution and mix well with magnet stirrer.
vii. Sterilize solution by filtration through 0.22 μm membrane.
viii. Aseptically divide to aliquots of 10 and/or 50 ml into sterile plastic tubes.
ix. Store in refrigerator at +4-C.
   b. BcS-LM
      BcS-LM is a special bacteria growth media for accelerated specific growth of Listeria.

### Reagents:

For preparing of 1 L and/or 0.5 L DGD-LM media needed the following reagents are described in Table 5:

TABLE 5
Composition of BcS-LM media

| | | | Amount | | | | | |
|---|---|---|---|---|---|---|---|---|
| [symbol] o | Reagent | Storage | 500 ml | | 1000 ml | | Cat. number | Manufacturer |
| 1 | Listeria Broth | RT | 375 | ml | 750 | ml | 84652.0500 | VWR Chemicals |
| 2 | DMEM (4.5 g)* | +4° C. | 100 | ml | 200 | ml | 11-055-1g | Biological Ind. |
| 3 | Fetal Calf Serum (FCS)* | −20° C. | 25 | ml | 50 | ml | 04-001-1A | Biological Ind. |
| 4 | Nalidixic acid | RT | 10 | mg | 20 | mg | 158542 | ALDRICH |
| 5 | Cycloheximide | RT | 10 | mg | 20 | mg | C4859 SIGMA | SIGMA |
| 6 | Galactose | +4° C. | 2.5 | g | 5 | g | G0625-100G | Sigma |
| 7 | Glucose | +4° C. | 1 | g | 2 | g | D8270-100G | Sigma |

*Unique additions for bacterial growth

2.2 Procedure
i. Prepare Listeria Broth according to the manufacturer's instructions:
ii. Dissolve 15 g in 1 L DDW with magnet stirrer;
iii. Add supplements for Listeria Broth and mix well with stirrer.
iv. Add DMEM, FCS, Galactose and Glucose. Mix well with magnet stirrer.
v. Sterilize solution by filtration through 0.22 μm membrane.
vi. Aseptically divide to aliquots of 10-50 ml into sterile plastic tubes.
vii. Store in refrigerator at 4-C.

TABLE 6
Composition of FCS (Fetal Calf Serum)
Composition of FBS

| Component | Average | Range |
|---|---|---|
| Endotxins (ng/ml) | 0.35 | 0.01-10.0 |
| Glucose (mg/ml) | 1.25 | 0.85-1.81 |
| Protein (mg/ml) | 38 | 32-70 |
| Albumin (mg/ml) | 23 | 20-36 |
| Hemoglobine (μg/ml) | 113 | 24-181 |
| Bilirubin, total (μg/ml) | 4 | 3-11 |
| Bilirubin, direct (μg/ml) | 2 | 0-5 |
| Urea (μg/ml) | 160 | 140-200 |
| Urate (μg/ml) | 29 | 13-41 |
| Creatinin (μg/ml) | 31 | 16-43 |
| Insulin (μU/ml) | 10 | 6-14 |
| Cortisol (ng/ml) | 0.5 | 0.1-23 |
| Growth hormone (ng/ml) | 39.0 | 18.7-51.6 |
| Parathormone, PTH (ng/ml) | 1.72 | 0.085-6.18 |
| Triiodothyronine, T3 (ng/ml) | 1.2 | 0.56-2.23 |
| Thyroxine, T4 (ng/ml) | 0.12 | 0.08-0.16 |
| Thyroid-stimulating hormone, TSH (ng/ml) | 1.22 | 0.2-4.5 |
| Follicle-stimulating hormone, FSH (pg/ml) | 95 | 20-338 |
| Testosterone (pg/ml) | 400 | 210-990 |
| Progesterone, P4 (pg/ml) | 80 | 3-360 |
| Prolactin = Luteotropic hormone, LTH (pg/ml) | 176 | 20-500 |
| Luteinizing hormone, LH ?? (pg/ml) | 8 | 1.2-18 |
| Prostaglandin E (ng/ml) | 5.9 | 0.5-30.5 |
| Prostaglandin F (ng/ml) | 12.3 | 3.8-42.0 |
| Vitamine A (ng/ml) | 90 | 10-350 |
| Vitamine E (ng/ml) | 1.1 | 1-4.2 |
| Cholesterol (μg/ml) | 310 | 120-630 |
| Lactate-dehydrogenase, LDH (mU/ml) | 864 | 260-1,215 |
| Alkaline Phosphatase (mU/ml) | 255 | 110-352 |
| Aspartate-Aminotransferase, ASAT (mU/ml) | 130 | 20-200 |

from Lindl, T. (2002): “Zell- und Gewebekultur”. 5th ed. Spektrum Akademischer Verlag, Heidelberg

According to Thermo's data, 111 ng/mil IGF, 12.6 ng/ml TGF-beta, 37.3 ng/ml FGF-2 is present in FBS. (However there are batch- to batch variations).

### Example 4

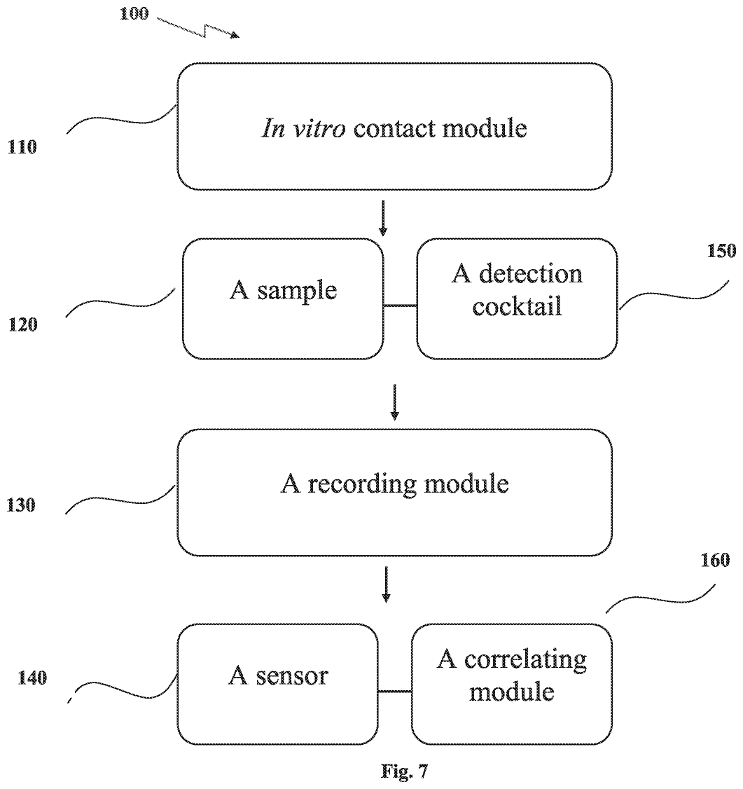

Reference is now made to FIG. 7, illustrating a system (100) useful for rapid detection and quantification of specific live and recovered microorganisms in a sample (120).

The system comprises:

a. an in vitro contact module (110) configured to contact the sample (120) with a detection cocktail, (150), the cocktail (150) comprises:
i. a nutrient medium comprising at least one of host-derived fraction or host sourced cell growth factors, for accelerated selective growing and multiplying of the microorganism;
ii. at least one fluorescent marker molecule for detection of intracellular metabolism by a sensor
iii. at least one metabolic activator for specifically increasing metabolism of said specific microorganism and increasing concentration of the fluorescent marker molecule into said specific microorganism;
b. a module for recording data (130) on the outcome of said in vitro contacting wherein the module comprises:
i. a sensor (140) for measuring gray levels of fluorescent intensity of the marker molecules; and
ii. a module for correlating (160) said detected gray levels to metabolism of the specific microorganism, determining the gray level to a predetermined threshold of metabolism; and further correlating the gray levels which are above the predetermined threshold with quantity of recovered high metabolic-active microorganism in the sample.

What is claimed is:

1. A method for rapid detection and quantification of mammalian pathogenic microorganisms in a sample, comprising steps of:
a. providing a detection cocktail comprising
i. a nutrient medium comprising at least one of a mammalian host-derived fraction, for accelerated selective growing and multiplying of said microorganism;
ii. at least one fluorescent marker molecule for detection of intracellular metabolism by a sensor
iii. at least one metabolic activator for specifically increasing metabolism of said specific microorganism and increasing intracellular concentration of said fluorescent marker molecule into said pathogenic microorganism;
b. contacting said sample with said detection cocktail;
wherein said detection and quantification is by steps of:
i. measuring gray levels of fluorescent intensity of said marker molecules by said sensor, said gray levels correlated to metabolism level of said pathogenic microorganism,
ii. determining said gray level to a predetermined threshold of metabolism;
further correlating said gray levels above said predetermined threshold with quantity of high metabolic-active said pathogenic microorganism in said sample, wherein said mammalian host-derived fraction is a mammalian tissue or cell.

2. The method of claim 1, wherein said mammalian pathogenic microorganism is selected from the group consisting of E. coli, Ps. aeruginosa and Listeria.

3. The method of claim 1, wherein Cetrimide and Fucidin are said metabolic activators when said mammalian pathogenic microorganism is Ps. aeruginosa.

4. The method of claim 1, wherein X-glucuronide or B-galactose or D-Glucose are said activators when said mammalian pathogenic microorganism is a Coliform bacteria.

5. The method of claim 1, wherein said mammalian host tissue is selected from the group consisting of nervous tissue, epithelial tissue, connective tissue, muscle tissue, adipose tissue, glandular tissue, organ tissue, blood, blood fractions, cerebrospinal fluid, urine, saliva, sweat and breast milk.

6. The method of claim 1, wherein said mammalian pathogenic microorganism is a human pathogenic microorganism.

7. The method of claim 1, wherein said mammalian pathogenic microorganism is a human pathogenic microorganism and said mammalian host derived fraction is a human host derived fraction.

8. The method of claim 1, wherein said detection cocktail further comprises a fluorescently labelled antibody complex configured to bind to cell wall antigens specific for recognizing said microorganisms.

9. The method of claim 1, wherein said microorganism is selected from the group consisting of bacteria, fungi, viruses, protozoa, yeasts, molds, parasites.

10. The method of claim 1, wherein said sample comprises at least one of fluid, water, food, beverage, blood, a solution, a pharmaceutical preparation, a mammalian sourced tissue and liquids, air, soil or surface.

11. The method of claim 1, wherein said metabolic activator is selected from a group consisting of sugars, proteins, effectors of membrane receptors, substrates of intercellular enzymatic reactions.

12. The method of claim 1, wherein said marker molecule is selected from a group consisting of conjugated antibodies, metabolized carbonates, DNA or RNA sequences, products of intracellular reactions, cell membrane parts, membrane receptors, specific effectors and extracellular liquids.

13. The method of claim 1, wherein said metabolic activator is selected from the group consisting of one of asparagine, beta-galactoside or NB medium.

14. The method of claim 1, wherein when said mammalian pathogenic microorganism is Ps. aeruginosa, said metabolic activator is Asparagine.

15. The method of claim 1, wherein when said mammalian pathogenic microorganism is Coliform bacteria, said metabolic activator is Beta-galactoside.

16. The method of claim 1, further including detecting and quantifying total bacteria in said sample, and wherein said metabolic activator for total bacteria is NB medium.

17. The method of claim 8, wherein said labelled antibody is selected from the group consisting of antibodies with ferromagnetic moieties, antibodies with paramagnetic moieties, antibodies with diamagnetic moieties, antibodies with fluorescent moieties, antibodies with phosphorescent moieties, antibodies with luminescent moieties, antibodies with electro-chemiluminescent moieties, chromatic moieties, antibodies with moieties having a detectable electron spin resonance, antibodies with electrical capacitance, antibodies with dielectric constant or antibodies with electrical conductivity.

18. The method of claim 8, wherein said labeled antibody complex comprises antibodies and antigens, further wherein antigens are selected from a group of amino acids, peptides, sugars, monosaccharides, polysaccharides and lipids.

19. The method of claim 1, further comprising a step of filtering via bacterial filter of arrange of 0.2 to 0.6 μm pore.

20. The method of claim 1, wherein time period for said detection of a specific organism in a sample is less than 24 hours.

21. The method of claim 1, further comprising steps of increasing the number of detectable microorganisms in said sample.

22. The method of claim 1, wherein number of said specific microorganism in said sample is equal or higher than the number of Colony Forming Units of said specific organism detected by conventional Hetero-Plate Count.

23. The method of claim 1, wherein said marker molecules are selected from the group consisting of Fluorescein, Fluorescein di(β-D-glucuronide), NileRed, and CY5.

24. The method of claim 8, wherein said antibody is selected from the group consisting of Anti-Pseudomonas aeruginosa monoclonal antibody and Anti-E. coli FITC.

25. The method of claim 1, wherein when said mammalian pathogenic microorganism is coliform bacteria, said nutrient medium is BcS-EC growth medium, comprising a mixture of Peptone water, McConkey Broth, DMEM, Fetal Calf Serum, 4-Nitrophenyl 3-D-glucuronide, Galactose and glucose.

26. The method of claim 1, wherein when said mammalian pathogenic microorganism is Listeria, said nutrient medium is BcS-LM growth medium comprises Listeria Broth, DMEM, Fetal Calf Serum, Nalidixic acid, Cycloheximide, Galactose, Glucose and FBS.
